(19) United States
(12) Patent Application Publication
(10) Pub. No.: US 2009/0099595 A1
(43) Pub. Date: Apr. 16, 2009

(54) Method and mixture for treating unilateral vocal fold paralysis

(75) Inventors: Henrich Cheng, Taipei (TW); Tung-Lung Tsai, Taipei (TW)

Correspondence Address: PANITCH SCHWARZE BELISARIO & NADEL LLP, ONE COMMERCE SQUARE, 2005 MARKET STREET, SUITE 2200, PHILADELPHIA, PA, 19103, US

(73) Assignee: Henrich CHENG, Taipei (TW)

(21) Appl. No.: 11/982,758

(22) Filed: Nov. 5, 2007

Related U.S. Application Data
(60) Provisional application No. 60/864,242, filed on Nov. 3, 2006.

(51) Int. Cl. A61L 24/00; A61L 24/02

(52) U.S. Cl. 606/214; 514/12; 424/682

(57) ABSTRACT

The present invention provides methods and mixtures for treating unilateral vocal fold paralysis (UVFP) and/or enhancing functional recovery of a damaged recurrent laryngeal nerve (RLN). The method of the present invention comprises applying a fibrin glue mixture to a portion of laryngeal muscle. The fibrin glue mixture comprises a growth factor, fibrinogen, aprotinin and divalent calcium ion.

### CROSS-REFERENCE TO RELATED APPLICATIONS

This application claims the benefit of U.S. Provisional Application No. 60/864,242, filed Nov. 3, 2006, the disclosure of which is incorporated herein by reference.

### FIELD OF THE INVENTION

The present invention relates to methods and mixtures for treating unilateral vocal fold paralysis (UVFP) and enhancing functional recovery of a damaged recurrent laryngeal nerve (RLN).

### BACKGROUND OF THE INVENTION

Neurological damage may limit functional outcome. For this reason, injuries to the nervous system require careful management to maximize recovery. The degree to which a nerve is damaged imposes substantial influence on its present function and potential for recovery. After complete axonal transection, the neuron undergoes a series of degeneration processes, followed by attempts at regeneration. The time-dependent decline of the ability of motoneuron to regenerate the axons after axotomy is one of the principal affecting factors to poor recovery after peripheral nerve injury, and the decline in neurotrophic support may be partially responsible for this effect.

Unilateral vocal fold paralysis (UVFP) is the loss of mobility of one vocal fold due to damage to the central or peripheral nervous system. Peripheral nervous system etiologies are more common and include damage to the vagus nerve or its recurrent laryngeal branch as the result of invasion or impingement on the nerve from neoplasms, accidental traumatic injury, neurologic disease or sacrifice of the nerve during skull base, neck, or cardio-thoracic surgeries. Central nervous system causes of neurological damage leading to UVFP are less common but might include stroke or other sources of intracranial lesions and neurologic diseases. Some causes of UVFP are unknown.

In some cases, the paralyzed vocal fold can be fixed relatively close to the midline. In such situations, the resulting voice disorder may be relatively mild. In other cases, the vocal fold is in a more lateral position, causing a wide glottic gap on phonation. With a larger glottic gap, the voice disorder is usually more severe. Altered vertical position and flaccidity in the paralyzed vocal fold also contribute to incomplete glottic closure and asymmetric vocal fold vibration. The resulting voice might be characterized by breathiness, hoarseness, roughness, diplophonia and reduced pitch and loudness dynamics. Considerable effort and fatigue are commonly experienced with voicing. Dysphagia and a weak cough may also be present with UVFP.

Interventions for UVFP fall into two primary categories—medical/surgical intervention and voice therapy. Medical/surgical interventions share the general objective of altering the shape, position or tonicity of the vocal fold. Even though the paralyzed vocal fold does not regain movement from these procedures, repositioning it towards midline or adjusting its vertical position reduces the glottic gap. This increases the potential for the functioning vocal fold to meet the paralyzed cord during phonation and even to entrain the paralyzed cord into vibration, producing a stronger and clearer voice. Voice therapy targets behavioral modifications that can optimize voice and communication function.

Medical/surgical interventions can be divided into three broad categories: injection augmentation, laryngeal framework surgery, and laryngeal reinnervation. In injection augmentation, substances such as collagen or autologous fat are injected into the paralyzed vocal fold to add bulk to the fold. This additional bulk might be adequate to close a small glottic gap. However, there is a risk of overmedialization or undermedialization, thus producing less than optimal voice. In addition, most injected materials may be resorbed within few weeks to months of injection.

Two types of laryngeal framework surgery are common for treating UVFP. In medialization thyroplasty (also called medialization laryngoplasty), a shim of silastic or other medical grade material is implanted into the paralyzed vocal fold through a surgically created window in the thyroid lamina. This shim is positioned such that it pushes the vocal fold towards the midline and fixes it there which can close a larger inter-membranous glottic gap. The second commonly used framework surgery is arytenoid adduction (AA). In this surgery, the arytenoid cartilage is rotated to simulate its position during normal adduction, and the cartilage is secured in this position by a suture. AA can assist in closing a large posterior glottic gap and in adjusting the vertical position of the vocal fold. Medialization thyroplasty and AA can be performed in isolation or in combination with each other. Laryngeal framework surgery is more invasive than injection augmentation, and it takes 6 to 12 months for spontaneous recovery or a stable paralytic cord position.

In laryngeal reinnervation, donor nerves such as the ansa cervicalis are brought to the paralyzed vocal fold to supply alternate enervation with the intended outcomes of reducing long-term atrophy, maintaining or improving muscle tone and bulk, stabilizing arytenoid cartilage positioning and perhaps enabling some tensing functions of laryngeal musculature. There are three options for nerve attachment: direct neuronal anastomosis, implanting a donor nerve in the laryngeal muscle, and nerve-muscle pedicle. While reinnervation does not appear to restore significant vocal fold adductory motion, the other adjustments from reinnervation can enhance both glottic closure and entrained vibratory behavior. It takes several months to obtain a stable result for laryngeal reinnervation, and there is a risk of synkinesis.

The first neuronal growth factor, nerve growth factor (NGF), discovered in the 1950s, promotes the survival and differentiation of sympathetic and sensory neurons. Many subsequent attempts were made to induce nerve regeneration at nerve defects using various neurotrophic factors: Glial-derived neurotrophic factor (GDNF) has a trophic effect on dorsal root gaglion cells as well as on motoneurons and autonomic neurons. Ciliary neurotrophic factor (CNTF) promotes survival of motoneurons in vitro and in neonatal animals following axotomy. Acidic fibroblast growth factor (aFGF) treatment prevents motoneuron loss, improves corticospinal tract regeneration, and contributes to angiogenesis in vitro.

Using insulin-like growth factor-I (IGF-I) in intralaryneal gene therapy has been proved effective in decreasing muscle atrophy and increasing muscle innervation in a series of rat animal studies. In addition, IGF-I intramuscular injection has been proved to decrease muscle atrophy after denervation in a rat leg muscle study (Day, C. S. et al., 2002, Microsurgery 22(4): 144-51).

Up to now, there has been no treatment for UVFP that can restore movement of the affected vocal cord. There remains a need for a means to effectively treat UVFP and to enhance the functional recovery of a damaged recurrent laryngeal nerve (RLN). The present inventor satisfies this need.

### BRIEF SUMMARY OF THE INVENTION

The present invention is based on the discovery that unilateral vocal fold paralysis (UVFP) can be effectively treated using a fibrin glue mixture to restore the function of the damaged recurrent laryngeal nerve (RLN).

Accordingly, one aspect of the invention relates to a method for treating UVFP comprising applying a fibrin glue mixture to a portion of laryngeal muscle, wherein the fibrin glue mixture comprises a growth factor, fibrinogen, aprotinin and divalent calcium ion.

Another aspect of the invention also relates to a method for enhancing the functional recovery of a damaged RLN comprising applying a fibrin glue mixture to a portion of laryngeal muscle, wherein the fibrin glue mixture comprises a growth factor, fibrinogen, aprotinin and divalent calcium ion.

In a preferred embodiment of the present invention, the fibrin glue mixture comprises an insulin-like growth factor (IGF), fibrinogen, aprotinin and divalent calcium ion.

In another preferred embodiment of the present invention, the laryngeal muscle is laryngeal adductor muscle.

### BRIEF DESCRIPTION OF THE SEVERAL VIEWS OF THE DRAWINGS

The patent or application file contains at least one drawing executed in color. Copies of this patent or patent application publication with color drawings will be provided by the Office upon request and payment of the necessary fee.

The foregoing summary, as well as the following detailed description of the invention, will be better understood when read in conjunction with the appended drawings. For the purpose of illustrating the invention, there is shown in the drawings an embodiment of the present invention.

In the drawings:

FIG. 1 shows the position of the fibrin glue cast in blue between thyroarytenoid (TA) and lateral cricoid arytenoid (LCA) muscles;

FIG. 2 shows the angle between the left vocal cord and the central line of larynx (i.e., the vertical yellow line in the photos) for both the study (“IGF-I”) and control (“Control”) groups;

FIG. 3 shows the waveform of the electric glottography (“EGG”) and artificial voice (“Voice”);

FIG. 4 shows the atrophy percentage of intralaryngeal muscles, including thyroarytenoid (TA), lateral cricoid arytenoid (LCA) and posterior cricoid arytenoid (PCA), for both the study (“IGF1”) and control (“control”) groups; and

FIG. 5 shows images of the histological staining of the lateral cricoarytenoid muscle for both the study (“IGF-1”) and control (“Control”) groups.

### DETAILED DESCRIPTION OF THE INVENTION

Unless defined otherwise, all technical and scientific terms used herein have the same meaning as commonly understood by one of ordinary skill in the art to which the invention pertains. Although any methods and materials similar or equivalent to those described herein can be used in the practice of testing of the present invention, the preferred materials and methods are described herein.

As used herein, the article “a” or “an” means one or more than one (that is, at least one) of the grammatical object of the article, unless otherwise made clear in the specific use of the article in only a singular sense.

For a better understanding of the present invention, some of the terms used herein are explained in more detail. It is also to be understood that the terminology used herein is for the purpose of describing particular embodiments only, and is not intended to be limiting. In describing and claiming the present invention, the following terminology will be used in accordance with the definitions set out below.

As used herein, the term “growth factor” includes a substance that promotes growth and development by directing cell maturation and differentiation and by mediating maintenance and repair of tissues. In particular embodiments, the growth factor can be any of a complex family of polypeptide biological factors. Each of the growth factors herein can be a natural substance produced by the body of an animal, including but not limited to, human, rat, mouse, pig, dog and monkey, or obtained from food, such as vitamins and minerals. It can also be a recombinant product produced by a recombinant host, such as a recombinant bacterium, from the growth factor gene.

A person skilled in the art will understand that each of the growth factors herein also includes a structural and/or functional derivative of the naturally occurring growth factor, such as a fragment of the growth factor or a chemically modified growth factor, that maintains the biological activity of the growth factor. The growth factor can be chemically modified to achieve certain desirable properties, such as enhanced stability or bioavailability. Common modifications to a protein include, for example, glycosylation, lipid attachment, sulfation, gamma-carboxylation of glutamic acid residues, hydroxylation and ADP-ribosylation.

As used herein, the term “fibrinogen” means a protein that is converted into fibrin by the action of thrombin especially during blood clot formation. The fibrinogen can be a natural substance produced in the liver of an animal, including but not limited to, human, rat, mouse, pig, dog and monkey. It can also be a recombinant product produced by a recombinant host, such as a recombinant bacterium, from the fibrinogen gene. The fibrinogen further includes any structural and/or functional derivative of the naturally occurring fibrinogen, such as a fragment of or a chemically modified fibrinogen, that maintains the biological activity of the fibrinogen.

As used herein, the term “aprotinin” refers to a polypeptide that is known for its protease-inhibiting properties, especially in inhibiting several serine proteases, such as trypsin, chymotrypsin, kallikrein, and pepsin. The aprotinin can be a natural substance produced in the organ of an animal, including but not limited to, human, rat, mouse, pig, dog and monkey. It can also be a recombinant product produced by a recombinant host, such as a recombinant bacterium, from the aprotinin gene. The aprotinin further includes any structural and/or functional derivative of the naturally occurring aprotinin, such as a fragment of or a chemically modified aprotinin that maintains the biological activity of the aprotinin.

The term “effective amount” as used herein, means that amount of a fibrin glue mixture that when applied to a portion of laryngeal muscle, more effectively treats unilateral vocal fold paralysis (UVFP) and/or enhances the functional recovery of a damaged recurrent laryngeal nerve (RLN), as compared to the treatment of UVFP or functional recovery of a damaged RLN without the application of the fibrin glue mixture.

Embodiments of the invention relate to methods for treating unilateral vocal fold paralysis (UVFP) and/or enhancing the functional recovery of a damaged recurrent laryngeal nerve (RLN) with an effective amount of a fibrin glue mixture comprising a growth factor, fibrinogen, aprotinin and divalent calcium ion. According to the invention, the components of the fibrin glue mixture can be simultaneously or separately applied to the appropriate portion of laryngeal muscle.

In embodiments of the invention, the growth factor contained in the fibrin glue mixture includes, but is not limited to, an insulin-like growth factor (IGF), a glial cell line-derived neurotrophic factor (GDNF), a transforming growth factor-beta, a fibroblast growth factor (FGF), a platelet-derived growth factor, an epidermal growth factor, a vascular endothelial growth factor (VEGF), a neurotrophin, or a combination of any two or more of the growth factors listed herein. In particular embodiments of the invention, the neurotrophin is selected from a nerve growth factor (NGF), a brain-derived neurotrophic factor (BDNF), a neurotrophin 3 (NT 3), a neurotrophin 4 (NT 5), a neurotrophin 5 (NT 4), and a combination of any two or more of the neurotrophins listed herein.

Preferably, the growth factor is an IGF, including an IGF-I, an IGF-II, and a combination thereof. Most preferably, the growth factor is an IGF-I.

According to the present invention, the divalent calcium ion in the fibrin glue mixture can be any physiologically acceptable calcium compound that dissociates in water and releases a divalent calcium ion, such as calcium chloride, calcium carbonate, or a combination thereof.

In a preferred embodiment of the present invention, the fibrin glue mixture comprises IGF, fibrinogen, aprotinin and divalent calcium ion. In a most preferred embodiment of the present invention, the fibrin glue mixture comprises IGF-I, fibrinogen, aprotinin and calcium chloride.

The concentration of the growth factor, such as IGF-I, in the fibrin glue mixture is preferably about 0.0001 milligram per milliliter (mg/ml) to about 1000 mg/ml of the total volume of the fibrin glue mixture, more preferably about 10 μg/ml to about 100 μg/ml. The concentration of fibrinogen is preferably about 0.1 mg/ml to about 1000 mg/ml of the total volume of the fibrin glue mixture, more preferably about 1 to about 50 mg/ml. The concentration of aprotinin is preferably about 10 Kilo International Unit per ml (KIU/ml) to about 2000 KIU/ml of the total volume of the fibrin glue mixture, more preferably about 500 KIU/ml to about 1500 KIU/ml. The concentration of the divalent calcium ion, such as calcium chloride, is preferably about 0.1 micromole (μmol) per milliliter (mM) of the total volume of the fibrin glue mixture, more preferably about 1 μmol/ml to about 20 μmol/ml.

In other embodiments, the fibrin glue mixture used in the present invention further comprises one or more additional substances for treatment of UVFP or repair of RLN, which is selected from, but is not limited to, the group consisting of a steroid, e.g. methylprednisone; a cytokine; a chemokine; a proteinase, e.g. a metalloproteinase; an extracellular matrix molecule, e.g. laminin or tenascin; a guidance molecule, i.e. a molecule that attracts or repels the migration of a cell, e.g. netrin, semaphorin, neural cell adhesion molecule, cadherin, thioredoxin peroxidase or Eph ligand; an anti-angiogenic factor, e.g. angiostatin, endostatin, TNP-470 or kringle 5; a neuroprotective agent, e.g. N-methyl D-aspartate (NMDA), a non-NMDA antagonist, a calcium channel blocker, nitric oxide synthase (NOS), a NOS inhibitor, peroxynitrite scavenger or a sodium channel blocker; and a Nogo gene polypeptide and antibodies that specifically bind to the polypeptide.

The fibrin glue mixture used in the method of the present invention may also optionally include a cell or cell suspension for facilitating repair, including but not limited to, Schwann cells, bone marrow cells, blood cells, stem cells and olfactory ensheathing glial (OEG) cells.

Another general aspect of the invention is a kit comprising a fibrin glue mixture that comprises growth factor, fibrinogen, aprotinin and divalent calcium ion, and instructions for using the fibrin glue mixture in treating unilateral vocal fold paralysis (UVFP). Yet another general aspect of the invention is a kit comprising a fibrin glue mixture that comprises growth factor, fibrinogen, aprotinin and divalent calcium ion, and instructions for using the fibrin glue mixture in enhancing the functional recovery of a damaged recurrent laryngeal nerve (RLN). Such kits can be combined in one or to be made and marketed separately. Such kits preferably comprise a compartmentalized carrier suitable to hold in close confinement at least one container containing the fibrin glue mixture. In preferred embodiments, the kit comprising a fibrin glue mixture that comprises IGF, fibrinogen, aprotinin and calcium chloride, and instructions for using the fibrin glue mixture in treating UVFP and/or enhancing the functional recovery of a damaged RLN.

The present invention is further illustrated by the following examples, which are provided for the purpose of demonstration rather than limitation.

### EXAMPLE 1

### Illustrative Fibrin Glue Mixtures

Exemplary fibrin glue mixtures that can be used in embodiments of the present invention are shown in Table 1.

TABLE 1

| Mixture No. | IGF-I | Fibrinogen | Aprotinin | CaCl₂ |
|---|---|---|---|---|
| Mixture 1 | 1 μg/ml | 50 mg/ml | 500 KIU/ml | 1 mM |
| Mixture 2 | 10 μg/ml | 30 mg/ml | 800 KIU/ml | 2 mM |
| Mixture 3 | 20 μg/ml | 20 mg/ml | 1000 KIU/ml | 5 mM |
| Mixture 4 | 50 μg/ml | 10 mg/ml | 1500 KIU/ml | 8 mM |
| Mixture 5 | 100 μg/ml | 1 mg/ml | 2000 KIU/ml | 10 mM |
| Mixture 6 | 66.7 μg/ml | 5 mg/ml | 1000 KIU/ml | 4 mM |

### EXAMPLE 2

### Animal Preparation and Surgery

All protocols are in accordance with the recommendations in the Guide for the Care and Use of Laboratory Animals published by the National Institute of Health, USA and approved by the Institutional Animal Care and Use Committee of the Taipei Veterans General Hospital. Under general anesthesia with spontaneous breathing, a vertical incision over the midline neck of dogs was done to expose left recurrent laryngeal nerve (RLN) and confirmed by nerve stimulator. The movements of vocal cords were recorded by telescope before and after transaction of left RLN. A 2 cm segment of RLN was removed and the cut end of RLN was ligated with 5-0 nylon and the wound was closed. The mixture of tissue glue and insulin-like growth factor-I (IGF-I) (group 1, N=6), or vehicle (group 2, N=6) were injected trans-orally into the left larynx from ventricle under direct vision by a double syringe injection system with a needle stopper to keep the depth of injection at about 7 mm. The injection was repeated immediately after the RLN section and every 2 weeks for 2 months. All the animals were kept 1 month after the last injection for further study.

### EXAMPLE 3

### Vocal Cord Position Study

The animals were under anesthesia with spontaneous breathing, and the movements of vocal cords were recorded by telescope. All the movements were recorded by a digital video recorder and the digital images were stored for further computer analysis.

### EXAMPLE 4

### Aerodynamic Study

Under general anesthesia, a tracheotomy was done. Another tracheal tube was placed into the upper cut end of trachea to the cricoid's level. The neck was incised in the middle line with the right recurrent laryngeal nerve exposed. Under electric stimulation, the left cords were closed to the midline of the glottis. A pressured airflow was sent from the upper tracheal tube to make the vocal cord vibration. Stroboscopy and electroglottography (EGG) were recorded for the analysis of cord mucosa wave and glottal gap when vibrated.

### EXAMPLE 5

### Histopathological Study

After the aerodynamic study, the larynxes were harvested for the study of 1) wet muscle weight measurement, 2) muscle fiber diameter analysis, 3) percentage of motor endplate contact with nerve in left cricoid arytenoid muscle and postercricoid arytenoids muscle. Cross sections were stained with hematoxyline and eosion for muscle fiber diameter analysis. Axial sections were stained to identify motor endplates and innervated nerves. Nonspecific antibody reactions were blocked with 2% BSA for 1 hr at room temperature. Sections were incubated overnight at 4° C. with a mouse anti-acetylcholinesterase (1:500; Chemicon) to identify motor endoplates and simultaneously with rabbit anti-NF-H (1:100; Santa Cruz) to identify neurofilaments. Sections were washed with PBS, incubated with Alex 488 fluorophore donkey anti-rabbit (1:200 Molecular Probes, Eugene, Oreg.) for 1 hr at room temperature and cyanine 3 donkey anti-mouse (1:200 Jackson ImmunoScientific, Inc.). The sections were then washed with PBS, mounted on uncoated slides, and covered with a coverslip with mounting medium H1000 (Vector Laboratories, Inc). Innervated motor endplate was visualized by merged image with yellow color. All the motor endplates were counted and the percentage of innervated motor endplate was calculated.

### EXAMPLE 6

### Statistical Analysis

Vocal cord position study: The area between two cords was measured by Image-J software. A comparison was done using one way t-test. Aerodynamic study: the analysis of right cord mucosa wave and glottal gap when vibration was performed using speech analysis software. A comparison was done using Chi-square test.

Histopathological study: Mean values of the lesser diameter of the muscle fiber, motor endplate length, and percentage of endplates with nerve contact in each animal were obtained. For statistical comparison of the means, t-test was performed. Statistical significance was set at P<0.05.

### EXAMPLE 7

### Selective Injection of IGF-I into Laryngeal Adductor Muscle for Treating Unilateral Vocal Fold Paralysis—Animal Study

Twelve dogs were anesthetized and their left recurrent laryngeal nerves were cut (denervation) after nerve stimulator confirmed. Two solutions were prepared: the A solution containing fibrinogen (10 mg/ml), aprotinin (2000 KIU/ml) and 133.3 μg/ml of IGF-I, and the B solution containing 8 μmol/ml of calcium chloride (8 mM). Two syringes, each containing 0.015 ml of one of the A and B solutions, respectively, were connected to a home-made connector so that the two solutions were mixed as a fibrin glue mixture just before injection into the muscle to form a glue cast.

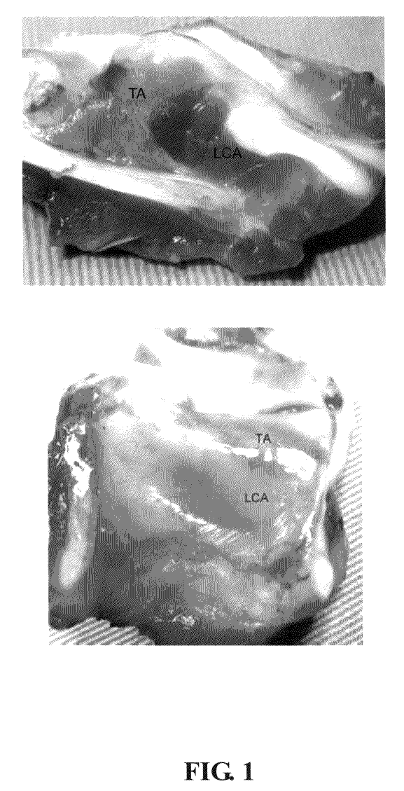

Immediately after denervation, 0.03 ml of the fibrin glue mixture obtained above (i.e. the mixture of the A solution and B solution) were injected into the left laryngeal adductor muscle by the double-syringe microinjection system (FIG. 1). Afterwards, the injection was performed every two weeks for 2 months. The 12 dogs were separated into two groups, each consisting of 6 dogs. The study group received 0.03 ml of the fibrin glue mixture per injection, containing 66.7 μg/ml of IGF-I in the mixture while the control group received the same fibrin glue mixture but without IGF-I.

At the end of the 12th week after denervation, studies were performed to investigate the static cord position, artificial vocal production, muscle atrophy percentage and histology for each dog. The statistic methods utilized were t-test in parametric data and Fisher's exact test in non-parametric data.

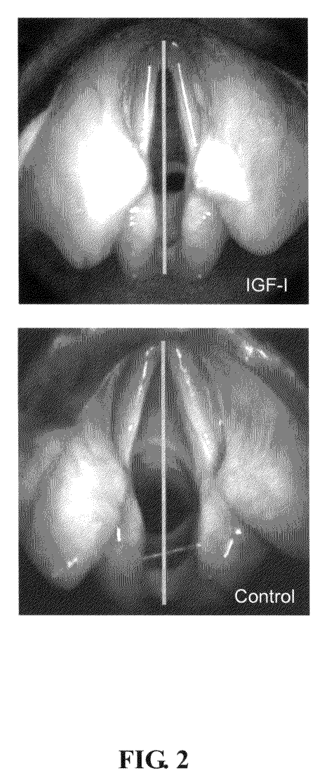

To investigate the static cord position, the angle between the left vocal cord and the central line of larynx (i.e., the line between the anterior and posterior commissure) was measured (FIG. 2). The mean angles of the left vocal cord were 16.51°±3.02° for the control group and 11.52°±2° in the study group (p=0.009).

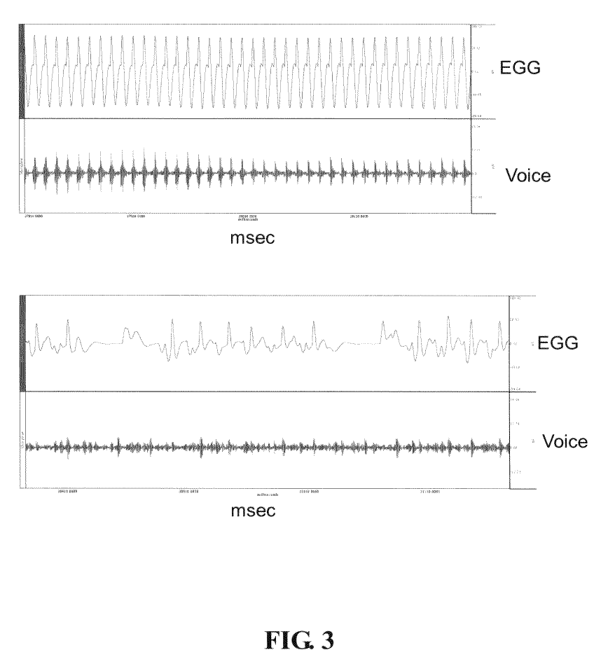

To investigate the artificial vocal production of the dogs, tracheostomy was performed and the laryngeal segment was connected to a compressed air source. The right recurrent laryngeal nerve was stimulated to adduct the right vocal cord, and voice was produced by the compressed air-flow driving. All the 6 dogs in the study group succeeded in artificial voice production with good mucosa wave, but only 2 dogs in the control group succeeded (p=0.03, one side) (FIG. 3). The EGG and voice from a study dog are shown in FIG. 3, top panel. The EGG and voice from a control dog were shown in FIG. 3, lower panel.

Investigation of the atrophy percentage of each intralaryngeal muscle and a histological study were performed after the animals were sacrificed. The atrophy percentage was obtained by the following formula:

$$1 - \frac{Left side wet muscle weight}{Right side wet muscle weight}$$

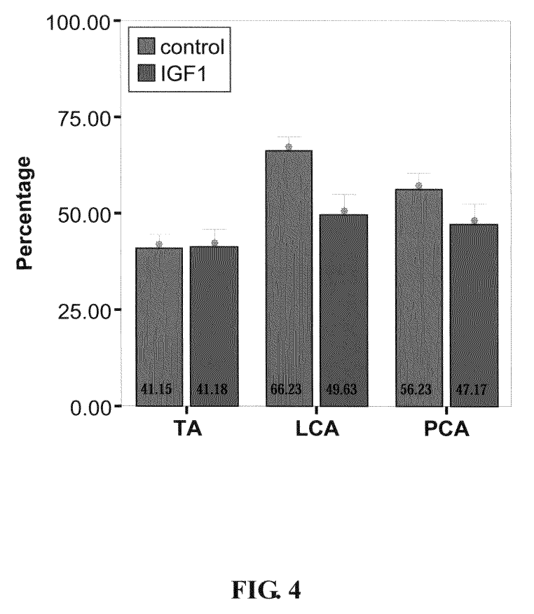

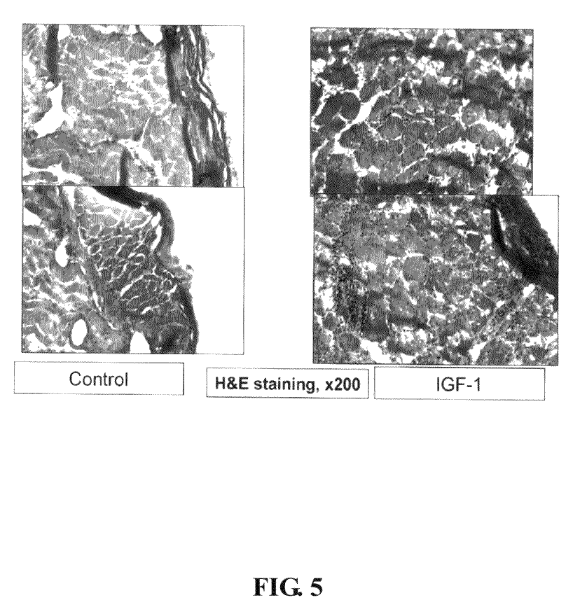

In the atrophy percentage, only the lateral cricoarytenoid muscle showed a significant difference between the two groups (study:control=49.63%:66.23%, p=0.026) (FIG. 4). The histological study was performed by H&E staining. The results are shown in FIG. 5.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A method for treating unilateral vocal fold paralysis (UVFP) comprising applying an effective amount of a fibrin glue mixture to a portion of laryngeal muscle, wherein the fibrin glue mixture comprises a growth factor, fibrinogen, aprotinin and divalent calcium ion.

2. The method of claim 1, wherein the growth factor is selected from the group consisting of an insulin-like growth factor (IGF), glial cell line-derived neurotrophic factor (GDNF), transforming growth factor-beta, fibroblast growth factor (FGF), platelet-derived growth factor, epidermal growth factor, vascular endothelial growth factor, neurotrophin, and combinations thereof.

3. The method of claim 2, wherein the growth factor is the IGF selected from the group consisting of an IGF-I, an IGF-II and a combination thereof.

4. The method of claim 3, wherein the IGF is the IGF-I.

5. The method of claim 1, wherein the laryngeal muscle is laryngeal adductor muscle.

6. The method of claim 1, wherein the divalent calcium ion is selected from the group consisting of calcium chloride, calcium carbonate and a combination thereof.

7. The method of claim 1, wherein the fibrin glue mixture comprises IGF, fibrinogen, aprotinin and calcium chloride.

8. The method of claim 1, wherein the fibrin glue mixture comprises IGF-I, fibrinogen, aprotinin and calcium chloride.

9. The method of claim 8, wherein the fibrin glue mixture comprises, per milliliter volume of the fibrin glue mixture, about 0.0001 to about 1000 mg of IGF-I, about 0.1 to about 1000 mg of fibrinogen, about 10 to 2000 KIU of aprotinin and about 0.1 to about 100 μmol of calcium chloride.

10. The method of claim 9, wherein the fibrin glue mixture comprises, per milliliter volume of the fibrin glue mixture, about 10 to about 100 μg of IGF-1, about 1 to about 50 mg of fibrinogen, about 500 to about 1500 KIU of aprotinin and about 1 to about 20 μmol of calcium chloride.

11. The method of claim 10, wherein the fibrin glue mixture comprises, per milliliter volume of the fibrin glue mixture, about 66.7 μg of IGF-1, about 5 mg of fibrinogen, about 1000 KIU of aprotinin and about 4 μmol of calcium chloride.

12. A method for enhancing the functional recovery of a damaged recurrent laryngeal nerve (RLN) comprising applying an effective amount of a fibrin glue mixture to a portion of laryngeal muscle, wherein the fibrin glue mixture comprises a growth factor, fibrinogen, aprotinin and divalent calcium ion.

13. The method of claim 12, wherein the growth factor is selected from the group consisting of an insulin-like growth factor (IGF), glial cell line-derived neurotrophic factor (GDNF), transforming growth factor-beta, fibroblast growth factor (FGF), platelet-derived growth factor, epidermal growth factor, vascular endothelial growth factor, neurotrophin, and combinations thereof.

14. The method of claim 13, wherein the growth factor is the IGF selected from the group consisting of an IGF-I, an IGF-II and a combination thereof.

15. The method of claim 14, wherein the IGF is the IGF-I.

16. The method of claim 12, wherein the laryngeal muscle is laryngeal adductor muscle.

17. The method of claim 12, wherein the divalent calcium ion is selected from the group consisting of calcium chloride, calcium carbonate and a combination thereof.

18. The method of claim 12, wherein the fibrin glue mixture comprises IGF, fibrinogen, aprotinin and calcium chloride.

19. The method of claim 12, wherein the fibrin glue mixture comprises IGF-I, fibrinogen, aprotinin and calcium chloride.

20. The method of claim 19, wherein the fibrin glue mixture comprises, per milliliter volume of the fibrin glue mixture, about 0.0001 to about 1000 mg of IGF-I, about 0.1 to about 1000 mg of fibrinogen, about 10 to 2000 KIU of aprotinin and about 0.1 to 100 μmol of calcium chloride.

21. The method of claim 20, wherein the fibrin glue mixture comprises, per milliliter volume of the fibrin glue mixture, about 10 to about 100 μg of IGF-1, about 1 to about 50 mg of fibrinogen, about 500 to 1500 KIU of aprotinin and about 1 to about 20 μmol of calcium chloride.

22. The method of claim 21, wherein the fibrin glue mixture comprises, per milliliter volume of the fibrin glue mixture, about 66.7 μg of IGF-1, about 5 mg of fibrinogen, about 1000 KIU of aprotinin and about 4 μmol of calcium chloride.

23. A kit comprising a fibrin glue mixture that comprises insulin-like growth factor (IGF), fibrinogen, aprotinin and divalent calcium ion, and instructions for using the fibrin glue mixture for treating unilateral vocal fold paralysis (UVFP) and/or for enhancing the functional recovery of a damaged recurrent laryngeal nerve (RLN).

24. The kit of claim 23, wherein the IGF is the IGF-I.

25. The kit of claim 24, wherein the fibrin glue mixture comprises, per milliliter volume of the fibrin glue mixture, about 0.0001 to about 1000 mg of IGF-I, about 0.1 to about 1000 mg of fibrinogen, about 10 to about 2000 KIU of aprotinin and about 0.1 to about 100 μmol of calcium chloride.
